(19) United States
(12) Patent Application Publication
(10) Pub. No.: US 2026/0364016 A1
(43) Pub. Date: Nov. 19, 2026

(54) Device for collecting particles of organic substances

(71) Applicant: Nanoform Finland Oyj, Helsinki (FI)

(72) Inventors: Ilkka Lassila, Helsinki (FI); Ari Kauppinen, Helsinki (FI); Tuomas Malve, Helsinki (FI); Peter Hänninen, Helsinki (FI); Tuomas Vainikka, Helsinki (FI); Gyan Awal, Helsinki (FI); Petteri Helander, Helsinki (FI); Serkan Gulsen, Helsinki (FI); Peter Blackburn, Helsinki (FI)

(21) Appl. No.: 19/121,807

(22) PCT Filed: Oct. 6, 2023
(86) PCT No.: PCT/FI2023/050573

§ 371 Date: Apr. 17, 2025

(30) Foreign Application Priority Data
Oct. 21, 2022 (FI) 20225956

(51) Int. Cl. A61K 9/16; A61K 45/06; B01D 7/00; H05B 3/02

(52) U.S. Cl. CPC A61K 9/1694; A61K 45/06; B01D 7/00; H05B 3/02

(57) ABSTRACT

The present disclosure relates to a device (200) for collecting particles of organic substances dispersed in solid carbon dioxide. The device comprises a collection chamber (201), at least one heatable and rotatable drum (202) and an opening (203). The drum is configured to heat to a temperature above −78 °C thereby sublimating the carbon dioxide and releasing the particles from the solid carbon dioxide, and to direct the released particles towards the collection chamber through the opening. The disclosure relates also to systems and methods for producing particles of organic substances wherein the particles are collected by using the device (200).

### FIELD

The present disclosure relates to devices for collecting particles of organic substances dispersed in solid carbon dioxide. The disclosure relates also to systems and methods for producing particles of organic substances wherein the particles are collected using the device.

### BACKGROUND

In the pharmaceutical industry, large number of drugs are insoluble or poorly soluble in water, which leads to a low dissolution rates and thus also low bioavailability of the drugs. One solution is to reduce particle size which leads to an improvement of the dissolution behavior. The Rapid Expansion of Supercritical Solution (RESS) is among the most used methods. Controlled Expansion of Supercritical Solutions (CESS) in turn, represents an improvement over RESS technologies due to the employment of controlled mass transfer, flow and pressure reduction.

In a typical RESS process, supercritical fluid is used to dissolve solid material under high pressure and temperature, thus forming a homogeneous supercritical phase. Thereafter, the solution is expanded through a nozzle and small particles are formed.

A system 100 suitable for producing and collecting of micro-and nanoparticles of organic substances using RESS and CESS technologies is shown in FIG. 1. The system comprises a pressure chamber 101 for a mixture of organic substance and supercritical carbon dioxide, a collection chamber 102, and an outlet tube 103 therebetween. The system comprises typically also one or more pressure controlling means 104 configured to control pressure within the system, and a nozzle 105 configured to allow expansion of the mixture from the outlet tube to the collection chamber.

When the mixture expands rapidly through the nozzle into ambient pressure, solid carbon dioxide comprising small particles of the organic substance is formed in the collection chamber. When the solid CO₂ formed sublimates, particles are freely distributed all around the collection chamber which may make the collection cumbersome. Significant amount of the particles may also exit the collection chamber via the exit vent 106 together with gaseous CO₂.

WO 2021/152204 discloses systems and a methods to produce particles of organic substances in the aid of dry ice formation wherein the particles are collected in the aid of an extension member engaged to a collection chamber and positioning a nozzle. Although the system and method disclosed therein prevents the exit of particles via the exit vent together with gaseous CO₂, there is still need for further devices and methods for collecting particles of organic substances.

### SUMMARY

It was observed that when particles of organic substances dispersed in solid carbon dioxide were allowed to sublimate on a rotating heated drum, and directing the released particles towards a collection chamber, one or more prior art problems of particle collection could be avoided or at least alleviated.

Accordingly, it is an object of the present disclosure to provide a new device for collecting particles of organic substances dispersed in solid carbon dioxide, the device comprising
a collection chamber,
one or more rotatable and heatable drums for positioning the particles of organic substances dispersed in solid carbon dioxide, and
one or more openings,
wherein
the one or more rotatable and heatable drums are configured to
   a) heat to a temperature above −78 °C thereby sublimating carbon dioxide and releasing the particles of the organic substances from the solid carbon dioxide, and
   b) direct the released particles towards the collection chamber through the one or more openings.

It is also an object of the present disclosure to provide a system for producing particles of organic substances comprising a device of claim 1 for collecting the particles of organic substances produced.

It is still an object of the present disclosure to provide a method for producing particles of organic substances wherein the particles are collected using the device of claim 1.

A number of exemplifying and non-limiting embodiments of the invention are described in accompanied dependent claims.

Various exemplifying and non-limiting embodiments of the invention and to methods of operation, together with additional objects and advantages thereof, are best understood from the following description of specific exemplifying embodiments when read in connection with the accompanying figures.

The verbs “to comprise” and “to include” are used in this document as open limitations that neither exclude nor require the existence of also unrecited features. The features recited in depending claims are mutually freely combinable unless otherwise explicitly stated. Furthermore, it is to be understood that the use of “a” or “an”, i.e., a singular form, throughout this document does not exclude a plurality.

### BRIEF DESCRIPTION OF FIGURES

The exemplifying and non-limiting embodiments of the invention are explained in greater detail below with reference to the accompanying figures, in which

FIG. 1 shows a system for producing particles of organic substance according to prior art,

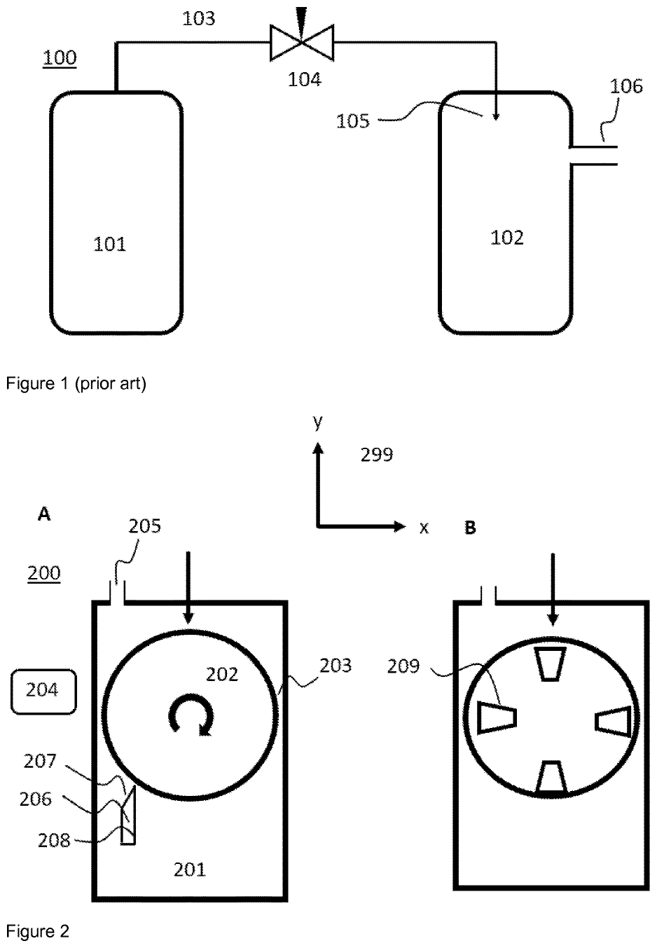

FIGS. 2, 3, 5 and 6 show devices for collecting particles of organic substance according to exemplary non-limiting embodiments of the present disclosure,

FIG. 4 demonstrates principle of collecting particles of organic substances (o) dispersed in solid carbon dioxide using an exemplary device of the present disclosure, and

FIG. 7 shows a system for producing and collecting particles of organic substances comprising a device according to an exemplary non-limiting embodiment of the present disclosure.

### DESCRIPTION

The specific examples provided in the description given below should not be construed as limiting the scope and/or the applicability of the appended claims. Lists and groups of examples provided in the description given below are not exhaustive unless otherwise explicitly stated.

According to one aspect the present disclosure concerns a device for collecting particles of organic substances dispersed in solid carbon dioxide, the device comprising
a collection chamber,
one or more rotatable and heatable drums for positioning the particles of organic substances dispersed in solid carbon dioxide, and
one or more openings,
wherein
the one or more rotatable and heatable drums are configured to
   a) heat to a temperature above −78 °C thereby sublimating carbon dioxide and releasing the particles of the organic substances from the solid carbon dioxide, and
   b) direct the released particles towards the collection chamber through the one or more openings.

The one or more rotatable and heatable drums are heated typically to a temperature between −78 °C and 40 °C, preferably to a temperature between 20 °C and 25 °C. Temperatures above ca 40 °C are not preferable if the organic substance is heat-sensitive. Typical speed of rotation is 0.05-5 rounds per second, such as 0.1-1 rounds per second.

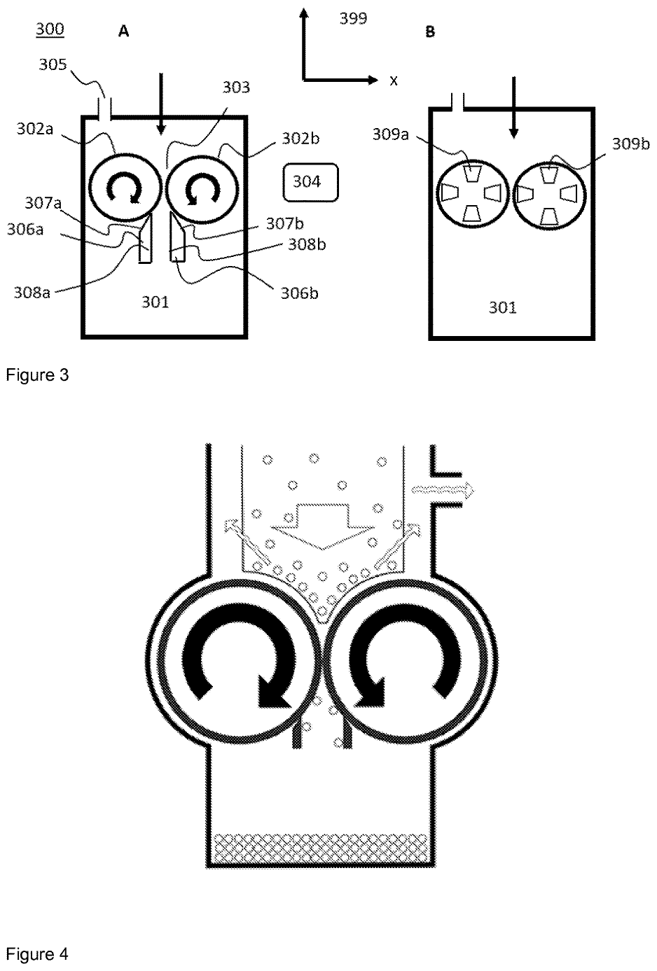

FIG. 2 illustrates a device 200 according to an exemplary and non-limiting embodiment of the present disclosure. The device comprises a collection chamber 201, a first rotatable and heatable drum 202 for positioning particles of organic substances dispersed in solid carbon dioxide, and an opening 203. Direction of rotation is shown in the figure as a circular arrow. The straight arrow presents a nozzle configured to be engaged to the device. The nozzle is for producing the particles of organic substances dispersed in solid carbon dioxide.

The first rotatable and heatable drum is configured to
a) heat to a temperature above −78 °C thereby sublimating carbon dioxide and releasing the particles of organic substances dispersed in the solid carbon dioxide, and
b) direct the released particles of organic substances towards the collection chamber through the opening.

Diameter of the opening in x-direction of the coordinate system 299 should be large enough to allow smooth direction of the particles towards the collection means but small enough to avoid penetration of solid carbon dioxide though the opening when the device is in operation. Exemplary diameter is from 1 um to 5 mm, such as 1 mm. Diameter of the opening must be larger than diameter of the particles.

The device 200 comprises heating means 204 for heating the drum above −78 °C to assist carbon dioxide sublimation. According to one embodiment the heating means comprises heat transfer fluid configured to circulate in drum. Exemplary heat transfer liquids are water and glycol. Also, gas flow can be used to heat transfer. According to another embodiment the heating means comprises electric heating elements positioned in contact to the drum.

When the device is in operation the rotating drum heats the CO₂ to a temperature above −78 °C thereby sublimating carbon dioxide, releasing the particles from the CO₂ and directs the released particles towards the collection chamber through the opening. The drum 202 is preferably made of thermally conductive material. An exemplary material is stainless steel. The device 200 comprises also an exit vent 205 for gaseous carbon dioxide. According to another embodiment the device is open so that an exit vent is not needed.

The released particles may retain on surface of the first drum. Thus, it is preferable that the device comprises means for removing particles from the surface of the first drum.

According to one embodiment the device comprises a scraping means 206 configured to remove released particles from the surface of the first drum. According to an exemplary embodiment the scarping means comprises a blade 207 in close proximity or in contact to the rotatable drum. The scraping means shown in the FIG. 2A comprises also guiding means 208 for directing the particles to the collection chamber.

According to another embodiment shown in FIG. 2B the device comprises means 209 for vibrating the first drum for removing the released particles from the surface of the first drum. Exemplary vibrating means is a mechanical vibrator. Another exemplary vibrating means is an ultrasound transducer. The device may comprise one or more vibration means. The exemplary device shown in FIG. 2B comprises four ultrasound transducers positioned inside the rotatable and heatable drum. The ultrasound transducers can be positioned also in proximity of the outer surface of the drum (not shown).

FIG. 3 shows a device 300 according to another embodiment of the present disclosure. The major difference between the device shown in FIG. 2 and FIG. 3 is that the device 300 comprises two rotatable and heatable drums. Accordingly, the device 300 comprises a collection chamber 301, a first rotatable and heatable drum 302a, a second rotatable and heatable drum 302b, and an opening 303. Direction of rotation is shown in the figure as circular arrows. The straight arrow presents a nozzle configured to be engaged to the device for producing the particles of organic substances dispersed in solid carbon dioxide to be collected.

The drums are adjacent each other, and the opening 303 is between the drums. Diameter of the opening in x-direction of the coordinate system 399 should be large enough to allow smooth direction of the particles towards the collection means but small enough to avoid penetration of solid carbon dioxide though the opening when the device is in operation. Exemplary diameter is from 1 μm to 5 mm, such as 1 mm. Size of the opening must be larger than the size of the particles.

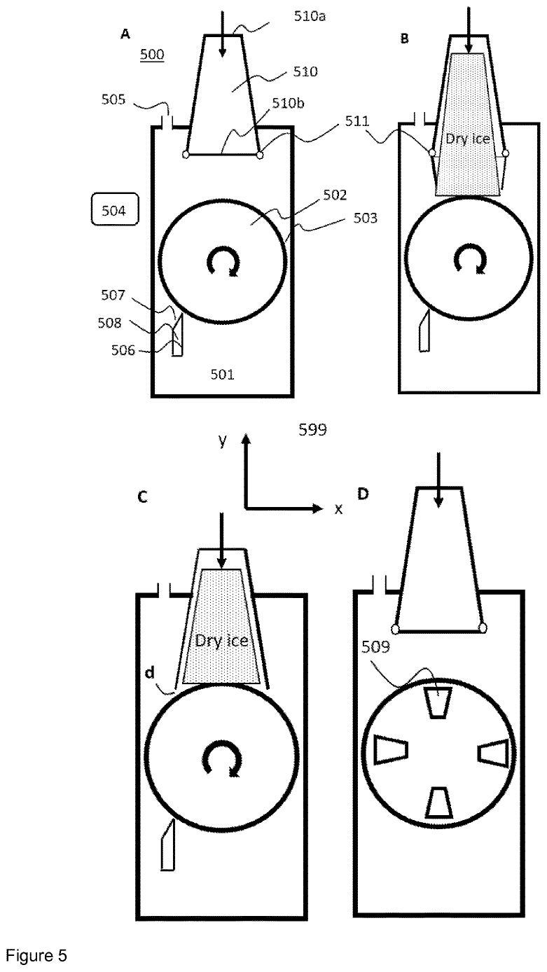

When the device comprises two drums, the first drum 302a is configured to rotate clockwise and the second drum 302b is configured to rotate anti-clockwise. The rotating motions push the particles of organic substances dispersed in solid carbon dioxide between the drums as shown in FIG. 4. Furthermore, since gaseous carbon dioxide (marked in the FIG. 4 by waved arrows) is forced to pass through the dry ice cake coming from above (moving direction marked by an arrow) the solid CO₂ filters any drug particles that may have ended up with the gas.

The device can include more than two rotatable and heatable drums, such as four, six or eight drums. The use of plurality of drums may be preferable when collection of large amount of particles of organic substances from solid carbon dioxide is needed. When the device comprises more than two drums it preferably also comprises more than two openings between the drums and the collection chamber.

The device 300 comprises heating means 304 for heating the drums above −78 °C to assist carbon dioxide sublimation. According to one embodiment the heating means comprises heat transfer fluid configured to circulate in drum. Exemplary heat transfer liquids are water and glycol. Also, gas flow can be used to heat transfer. According to another embodiment the heating means comprises an electric heating elements positioned in contact to the drum.

When the device is in operation the rotating drums heat the CO₂ to a temperature above −78 °C thereby sublimating carbon dioxide and releasing the particles from the CO₂ and direct the released particles towards the collection means through the opening. The drums 302a, b are preferably made of thermally conductive material. An exemplary material is stainless steel. The device 300 comprises also an exit vent 305 for gaseous carbon dioxide. According to another embodiment the device is open so that an exit vent is not needed.

The particles may retain on surface of the first and the second drum. Thus, it is preferable that the device comprises means for removing particles from the surface of the first drum.

According to one embodiment the device comprises a scraping means 306a, b configured to remove released particles from the surface of the first drum and from the surface of the second drum. According to an exemplary embodiment the scarping means comprises a blade 307a, b in close proximity or in contact to the rotatable drums. The scraping means shown in the FIG. 3A comprises also guiding means 308a, b for directing the particles towards the collection chamber.

According to the embodiment shown in FIG. 3B the device comprises means 309a, b for vibrating the first drum and the second for removing the released particles from the surface of the drums. Exemplary vibrating means is a mechanical vibrator. Another exemplary vibrating means is an ultrasound transducer. The device may comprise one or more vibration means. The exemplary device shown in FIG. 3B comprises four ultrasound transducers positioned inside the rotatable and heatable drum. The ultrasound transducers can be positioned also in proximity of the outer surface of the drum (not shown).

FIG. 5 illustrates a device 500 according to another an exemplifying and non-limiting embodiment of the present disclosure. The device comprises
a collection chamber 501,
an extrusion cone 510 for particles of organic substances dispersed in solid carbon dioxide, the extrusion cone comprising a first end 510a for positioning a nozzle and a second end 510b,
a first rotatable and heatable drum 502 positioned between the extrusion cone and the collection means, and
an opening 503.

The nozzle is marked in the figure by an arrow. The device comprises also an exit port 505 for gaseous carbon dioxide.

The second end of the extrusion cone is configured to allow release of the particles of organic substances dispersed in solid carbon dioxide from the extrusion cone onto the first rotatable and heatable drum. The first rotatable and heatable drum is configured to
a) heat to a temperature above −78 °C thereby sublimating carbon dioxide and releasing the particles of organic substances dispersed in the solid carbon dioxide, and
b) direct the released particles of organic substances towards the collection chamber through the opening.

Diameter of the opening in x-direction of the coordinate system 599 should be large enough to allow smooth direction of the particles towards the collection means but small enough to avoid penetration of solid carbon dioxide though the opening when the device is in operation. Exemplary diameter is from 1 μm to 5 mm, such as 1 mm. Size of the opening must be larger than the size of the particles.

The device 500 comprises heating means 504 for heating the drum above −78 °C to assist carbon dioxide sublimation. According to one embodiment heating means comprises heat transfer fluid configured to circulate in drum. Exemplary heat transfer liquids are water and glycol. Also, gas flow can be used to heat transfer. According to another embodiment the heating means comprises an electric heating elements positioned in contact to the drum.

When the device is in operation the rotating drum heats the CO₂ to a temperature above −78 °C thereby sublimating carbon dioxide and releasing the particles from the CO₂ and direct the released particles towards the collection means through the opening. The drum 502 is preferably made of thermally conductive material. An exemplary material is stainless steel. The device 500 comprises also an exit vent 505 for gaseous carbon dioxide. According to another embodiment the device is open so that an exit vent is not needed.

According to one embodiment the second end of the extrusion cone comprises a shutter 511. An exemplary shutter comprises one or more metal plates hold close by a spring force. According to this embodiment the extrusion cone is configured to hold solidifying carbon dioxide comprising the particles of organic substances until solid carbon dioxide comprising the particles of the organic substances fills the extrusion cone, after which the shutter is forced open by the growing volume of solid carbon dioxide. This allows the particles of organic substances dispersed in solid carbon dioxide to move towards the first drum. A situation where the shutter is forced open by a block of dry ice comprising the particles of organic substances is shown in FIG. 5B. The movement towards the drum is assisted by gravity when the device is its operating position.

An advantage of the extrusion cone comprising the shutter at the second end is that it allows the formation of a block of solid carbon dioxide comprising the dispersed particles of organic substances in the extrusion cone and release the block only after its size exceeds the size of the extrusion cone. This allows filling the part of the device between the one or more rotatable and heatable drums and the extrusion cone as effectively as possible by the organic substances dispersed in solid carbon dioxide. This, in turn prevents exit of the particles of organic substances with caseous carbon dioxide as shown in FIG. 4.

According to another embodiment the second end of the extrusion cone does not have a shutter. According to this embodiment the second end is positioned in close proximity i.e., distance d between the second end and first drum is e.g., from 1 μm-5 mm, such as 1 mm. Thus, the carbon dioxide is configured to solidify before the first drum as shown in FIG. 5C.

The particles may retain on surface of the first drum. Thus, it is preferable that the device comprises means for removing particles from the surface of the first drum.

According to one embodiment the device 500 comprises a scraping means 506 configured to remove released particles from the surface of the first drum. According to an exemplary embodiment the scarping means comprises a blade 507 in close proximity or in contact to the rotatable drum. The scraping means shown in the FIG. 5A comprises also guiding means 506 for directing the particles towards the collection chamber.

According to another embodiment the device comprises means 509 for vibrating the first drum for removing the released particles from the surface of the first drum. Exemplary vibrating means is a mechanical vibrator. Another exemplary vibrating means is an ultrasound transducer. The device may comprise one or more vibration means. The exemplary device shown in FIG. 5D comprises four ultrasound transducers positioned inside the rotatable and heatable drum. The ultrasound transducers can be positioned also in proximity of the outer surface of the drum (not shown).

FIG. 6 shows a device 600 according to still another embodiment of the present disclosure. The major difference between the device shown in FIG. 5 is that the device 600 comprises two rotatable and heatable drums. Accordingly, the device 600 comprises
a collection chamber 601,
an extrusion cone 610 for the particles of organic substances dispersed in solid carbon dioxide, the extrusion cone comprising a first end 610a for positioning a nozzle and a second end 610b,
a first rotatable and heatable drum 602a positioned between the extrusion cone and the collection chamber,
a second rotatable and heatable drum 602b positioned between the extrusion cone and the collection chamber, and
an opening 603.

The nozzle is marked in the figure by an arrow. The device comprises also an exit port 605 for gaseous carbon dioxide.

The drums are adjacent each other and positioned between the extrusion cone and the collection chamber. The second end of the extrusion cone is configured to allow release of the particles of organic substances dispersed in solid carbon dioxide from the extrusion cone onto the first rotatable and heatable drum, and onto the second rotatable and heatable drum. According to this embodiment the opening 603 is between the drums. Diameter of the opening in x-direction of the coordinate system 699 should be large enough to allow smooth direction of the particles towards the collection means but small enough to avoid penetration of solid carbon dioxide though the opening when the device is in operation. Exemplary diameter is from 1 μm to 5 mm, such as 1 mm. Size of the opening must be larger than the size of the particles.

When the device comprises two drums, the first drum 602a is configured to rotate clockwise and the second drum 602b is configured to rotate anti-clockwise. The rotating motions pushes the particles of organic substances dispersed in solid carbon dioxide between the drums.

The device 600 comprises heating means 604 for heating the drums above −78 °C to assist CO₂ sublimation. According to one embodiment the heating means comprises heat transfer fluid configured to circulate in the first rotatable drum and in the second rotatable drum. Exemplary heat transfer liquids are water and glycol. Also, gas flow can be used to heat transfer. According to another embodiment the heating means comprises an electric heating elements positioned in contact to the first and the second drum.

The drums 602a, b are preferably made of thermally conductive material. An exemplary material is stainless steel. The device 600 comprises also an exit vent 605 for gaseous carbon dioxide. According to another embodiment the device is open so that an exit vent is not needed.

According to one embodiment the second end of the extrusion cone comprises a shutter 611. An exemplary shutter comprises one or more metal plates hold close by a spring force. According to this embodiment the extrusion cone is configured to hold solidifying carbon dioxide comprising the particles until solid carbon dioxide comprising the particles of the organic substances fills the extrusion cone, after which the shutter is forced open by the growing volume of solid carbon dioxide. This allows the particles of organic substances dispersed in solid carbon dioxide to move towards the first drum and the second drum.

According to another embodiment the second end of the extrusion cone does not have a shutter. According to this embodiment the second end is positioned in close proximity of the drums, i.e., distance between the second end and first drum is from 1 μm-5 mm, such as 1 mm, and distance the second end and second drum is from 1 μm-5 mm, such as 1 mm. Thus, the carbon dioxide is configured to solidify before the first drum and the second drum. Accordingly, filling up of the extrusion cone with dry ice is enabled by the first drum and the second drum.

The particles may retain on surfaces of the first drum and the second drum. Thus, it is preferable that the device comprise means for removing particles from the surfaces.

According one embodiment the device comprises a scraping means 606a, b configured to remove released particles from the surface of the first drum 602a and from the surface of the second drum 602b, respectively. According to an exemplary embodiment the scarping means comprises blade 607a, b in close proximity or in contact to the rotatable drums. The scraping means shown in the FIG. 6A comprises also guiding means 608a, b for directing the particles towards the collection chamber.

According to another embodiment the device 600 comprises means 609a, b for vibrating the first drum and the second drum for removing the released particles from surface of the first drum and the second drum. Exemplary vibrating means is a mechanical vibrator. Another exemplary vibrating means is an ultrasound transducer. The device may comprise one or more vibration means. The exemplary device shown in FIG. 6B comprises four ultrasound transducers positioned inside the rotatable and heatable drums. The ultrasound transducers can be positioned also in proximity of the outer surface of the drums (not shown).

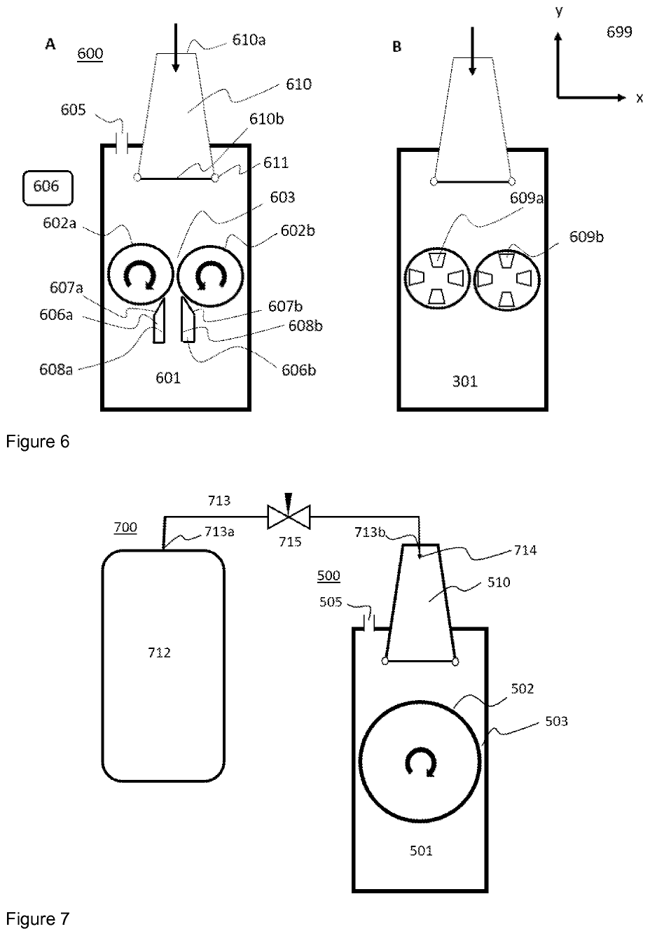

According to another aspect the present disclosure concerns a system for producing and collecting particles of organic substances. The system comprises means for producing the particles of organic substances dispersed in sold carbon dioxide, and a device of the present disclosure for collecting the particles. An exemplary system 700 engaged to the device 500 is shown in FIG. 7 as an illustrative example. Accordingly, the system comprises
a pressure chamber 712 for a mixture of organic substance and supercritical carbon dioxide,
an outlet tube 713 comprising a first end 713a engaged to the pressure chamber and a second end 713b comprising a nozzle 714, and
a device 500 for collecting the particles of the organic substances engaged to the nozzle.

The system comprises preferably also one or more means 715 configured to control pressure in the system and an exit port for gaseous carbon dioxide. The device 500 comprises also an exit vent 305 for gaseous carbon dioxide.

According to still another aspect the present disclosure concerns a method for producing particles of organic substances, wherein the method comprises the following steps
a) admixing organic substance with supercritical carbon dioxide in a pressure chamber 712 to form a mixture at a first pressure (P₁),
b) passing the mixture through an outlet tube 713 toward the nozzle,
c) allowing the mixture to expand through a nozzle 714 to an extrusion cone 510 at a final pressure (P_{F}) thereby forming the particles of organic substances dispersed in solid carbon dioxide,
d) positioning the particles of organic substances dispersed in solid carbon dioxide from the extrusion cone on a rotatable and heatable drum 502,
e) heating the drum at least to −78 °C thereby sublimating the carbon dioxide and releasing the particles of organic substances,
f) rotating the one or more rotatable and heatable drums thereby directing the released particles through an opening 503 towards a collection chamber 501, and
g) collecting the particles.

According to a preferable embodiment the collecting comprises scraping particles from surface of the rotating first drum and also, if present, on surface of the rotating second drum.

According to other preferable embodiment the collecting comprises vibrating the rotating drum(s) thereby releasing particles from the surface(s) of the drum(s).

According to a particular embodiment the step b) comprises decreasing the first pressure to a second pressure (P₂) during the passing. The decreasing is preferably gradual.

According to a particular embodiment step c) comprises decreasing the second pressure to the final pressure (P_{F}). The ratio (P₁)/(P₂) and (P₂/P_{F}) is preferably <15, more preferably <10.

The final pressure is typically atmospheric pressure.

Average particle size of organic substances obtained by the method of the present disclosure is typically 900 nm or less, preferably less than 500 nm, more preferably less than 100 nm, and most preferably less than 20 nm.

As defined herein an “organic substance” is a molecule containing carbon, excluding carbon containing alloys, and relatively small number of carbon-containing compounds such as metal carbonates and carbonyls, simple oxides of carbon and cyanides, as well as allotropes of carbon and simple carbon halides and sulfides which are considered inorganic. Exemplary organic substances used in the present disclosure are active pharmaceutical ingredients including medicaments and their pharmaceutically acceptable organic and inorganic salts.

A non-limiting list of exemplary classes of biologically active materials are active pharmaceutical ingredients that may be of interest include analgesics, antagonists, anti-inflammatory agents, anthelmintics, antianginal agents, antiarrhythmic agents, antibiotics (including penicillins), anticholesterols, anticoagulants, anticonvulsants, antidepressants, antidiabetic agents, antiepileptics, antigonadotropins, antihistamines, antihypertensive agents, antimuscarinic agents, antimycobacterial agents, antineoplastic agents, antipsychotic agents, immunosuppressants, antithyroid agents, antiviral agents, antifungal agents, anxiolytic sedatives (hypnotics and neuroleptics), astringents, beta-adrenoceptor blocking agents, blood products and substitutes, anti-cancer agents, cardiacinotropic agents, contrast media, corticosterioids, cough suppressants (expectorants and mucolytics), diuretics, dopaminergics (antiparkinsonian agents), haemostatics, immunosuppressive and immunoactive agents, lipid regulating agents, muscle relaxants, parasympathomimetics, parathyroid calcitonin and biphosphonates, prostaglandins, radiopharmaceuticals, sex hormones (including steroids), anti-allergic agents, stimulants and anorexics, sympathomimetics, thyroid agents, vasodilators, neuron blocking agents, anticholinergic and cholinomimetic agents, antimuscarinic and muscarinic agents, vitamins, and xanthines.

The organic substances, such active pharmaceutical ingredient, may be crystallic, amorphic or their mixtures. According to one embodiment, the organic substances comprise active pharmaceutical ingredient and one or more excipients.

The specific examples provided in the description given above should not be construed as limiting the scope and/or the applicability of the appended claims.

1. A device for collecting particles of organic substances dispersed in solid carbon dioxide, the device comprising:
a collection chamber,
one or more rotatable and heatable drums positioned above the collection chamber, when the device is in its operational position, for positioning the particles of organic substances dispersed in solid carbon dioxide,
an extrusion cone, positioned above the one or more rotatable and heatable drums, for the particles of organic substances dispersed in solid carbon dioxide, the extrusion cone comprising a first end for positioning a nozzle and a second end, and
one or more openings between the one or more rotatable and heatable drums and the collection chamber,
wherein the second end of the extrusion cone is configured to allow directing the particles of organic substances dispersed in solid carbon dioxide from the extrusion cone onto the one or more heatable and rotatable drums, and wherein the one or more rotatable and heatable drums are configured to
a) heat to a temperature above −78 °C thereby sublimating carbon dioxide and releasing the particles, and
b) direct the released particles towards the collection chamber through the one or more openings.

2. The device according to claim 1, wherein the diameter of the one or more openings is larger than the diameter of the particles but ≤5 mm.

3. The device according to claim 1, further comprising scraping means and/or vibration generating means, configured to remove particles from a surface of the one or more heatable and rotatable drums when the device is in operation.

4. The device according to claim 3, wherein the scraping means comprise a blade adjacent to the surface of the one or more heatable and rotatable drums.

5. The device according to claim 3, wherein the vibration generating means is selected from a mechanical vibrator and an ultrasound transducer.

6. The device according to claim 1, wherein the second end is open and wherein the distance between the second end and the one or more heatable and rotatable drums is 1 mm-5 mm.

7. The device according to claim 1, wherein the second end comprises a shutter configured to allow closing and opening of the extrusion cone.

8. A system for producing and collecting particles of organic substances, the system comprising:
a pressure chamber for a mixture of organic substance and supercritical carbon dioxide,
an outlet tube comprising a first end engaged to the pressure chamber and a second end comprising a nozzle, and
a device according to claim wherein the nozzle is engaged to the first end of the extrusion cone of said device.

9. A method for producing and collecting particles of organic substances using a system according to claim 8, the method comprising the steps of:
a) admixing organic substance with supercritical carbon dioxide in the pressure chamber to form a mixture at a first pressure (P₁),
b) passing the mixture through the outlet tube towards the nozzle,
c) allowing the mixture to expand through the nozzle to a final pressure (P_{F}) thereby forming the particles of organic substances dispersed in solid carbon dioxide,
d) positioning the particles of organic substances dispersed in solid carbon dioxide on one or more rotatable and heatable drums,
e) heating the one or more rotatable and heatable drums at least to −78 °C thereby sublimating the carbon dioxide and releasing the particles of organic substances,
f) rotating the one or more rotatable and heatable drums thereby directing the released particles through one or more openings towards a collecting means, and
g) collecting the particles of organic substances.

10. The method according to claim 9, wherein step b) comprises decreasing the first pressure (P₁) to a second pressure (P₂) during the passing.

11. The method according to claim 10, wherein the decreasing is gradual.

12. The method according to claim 10, wherein step c) comprises decreasing the second pressure (P₂) to the final pressure (P_{F}).

13. The method according to claim 10, wherein the second pressure (P₂) is less than 100 bar.

14. The method according to claim 9, wherein the organic substance is an active pharmaceutical ingredient.

15. The device according to claim 1, wherein the diameter of the one or more openings is 1 μm to 5 mm.

16. The method according to claim 10, wherein the second pressure (P₂) is 10-50 bar.

17. The device according to claim 1, wherein the one or more rotatable and heatable drums are configured to heat to a temperature between −78 °C and 40 °C.

18. The device according to claim 1, wherein the one or more rotatable and heatable drums comprise a thermally conductive material.

19. The method according to claim 10, wherein the ratios (P₁)/(P₂) and (P₂)/(P_{F}) are less than 10:1.

20. The method according to claim 9, wherein the average particle size of the particles of organic substances is 900 nm or less.
